(19) United States
(12) Patent Application Publication
(10) Pub. No.: US 2023/0414358 A1
(43) Pub. Date: Dec. 28, 2023

(54) SYSTEMS, METHODS, AND APPARATUSES FOR HEART VALVE REPAIR

(71) Applicant: Edward Lifesciences Innovation (Israel) Ltd., Caesarea (IL)

(72) Inventor: Sam Shafigh, Irvine, CA (US)

(21) Appl. No.: 18/446,369

(22) Filed: Aug. 8, 2023

Related U.S. Application Data
(63) Continuation of application No. PCT/IB22/50569, filed on Jan. 23, 2022.
(60) Provisional application No. 63/147,431, filed on Feb. 9, 2021.

(51) Int. Cl. A61F 2/24

(52) U.S. Cl. CPC A61F 2/2466; A61F 2220/0016

(57) ABSTRACT

Systems, apparatuses, and methods disclosed herein can be provided for medical treatment, including transcatheter medical treatments and/or functional mitral valve regurgitation within a human heart. The treatments may include reshaping a native annulus of the patient's heart, including the mitral valve and the tricuspid valve. The systems, apparatuses, and methods disclosed herein can include an anchor dispensing apparatus including a plurality of dispenser arms configured to extend radially outward from a central portion of the dispensing apparatus. A plurality of anchors can be provided, with one or more contraction members for cinching the plurality of anchors. A plurality of spacer bodies can be used to space the plurality of anchors from each other when the plurality of anchors are anchored to tissue.

### CROSS-REFERENCE TO RELATED APPLICATIONS

This application is a continuation of International Application No. PCT/IB2022/050569, filed Jan. 23, 2022, which designates the United States and was published in English by the International Bureau on Aug. 18, 2022 as WO2022/172108, which claims the benefit of U.S. Provisional Application No. 63/147,431, filed Feb. 9, 2021, the entire contents of each of which are hereby incorporated by reference for all purposes.

### BACKGROUND

Heart failure can occur when a ventricle of the heart becomes enlarged and dilated as a result of one or more of various etiologies. Initial causes of heart failure can include chronic hypertension, myocardial infarction, valve incompetency, and other dilated cardiomyopathies. With each of these conditions, the heart is forced to overexert itself in order to provide a cardiac output demanded by the body during various demand states. A dilated or enlarged heart and ventricle can result from or be associated with these conditions.

A dilated or enlarged heart, and particularly a dilated or enlarged left ventricle, can significantly increase tension and stress in heart walls both during diastolic filling and systolic contraction, which contributes to further dilatation or enlargement of chambers of the heart. In addition, valve incompetency or valvular regurgitation is a common comorbidity of congestive heart failure. As the dilation of a ventricle increases, valve function generally worsens, which results in a volume overload condition. The volume overload condition further increases ventricular wall stress, thereby advancing the dilation process, which further worsens valve dysfunction.

In heart failure, the size of the valve annulus (e.g., the mitral valve annulus, tricuspid valve annulus, etc.) may increase while the area of the leaflets of the valve remains constant. This can lead to reduced coaptation area between the valve leaflets, and, as a result, eventually to valve leakage or regurgitation. Moreover, in normal hearts, the annular size contracts during systole, aiding in valve coaptation. In heart failure, there can be poor ventricular function and elevated wall stress. These conditions tend to reduce annular contraction and distort annular size, often exacerbating valve regurgitation. In addition, as the chamber dilates, the papillary muscles (to which the leaflets are connected via the chordae tendineae) may move radially outward and downward relative to the valve, and relative to their normal positions. During this movement of the papillary muscles, however, the various chordae lengths remain substantially constant, which can limit the full closure ability of the leaflets by exerting tension on the leaflets. This condition is commonly referred to as “chordal tethering.” Annular changes and papillary changes can result in a poorly functioning valve.

Improved systems, methods, and apparatuses for heart valve repair, and anchoring systems generally, are accordingly desirable.

### SUMMARY

This summary is meant to provide some examples and is not intended to be limiting of the scope of the invention in any way. For example, any feature included in an example of this summary is not required by the claims, unless the claims explicitly recite the features. Also, the features, components, steps, concepts, etc. described in examples in this summary and elsewhere in this disclosure can be combined in a variety of ways. Various features and steps as described elsewhere in this disclosure may be included in the examples summarized here.

Systems, apparatuses, and methods disclosed herein can be used for medical treatment, including transcatheter medical treatments, such as treating valvular regurgitation within a human heart. The treatments can include reshaping a native annulus of the patient's heart, including the mitral valve, or the tricuspid valve, or another valve or structure of the human body. The systems, apparatuses, and methods disclosed herein can further be utilized for other forms of medical treatment and can be utilized for anchoring in other portions of a patient's body.

The systems, apparatuses, and methods disclosed herein can be utilized in a minimally invasive procedure, to access the heart without requiring a full sternotomy. Similarly, the systems, apparatuses, and methods disclosed herein can be utilized in a percutaneous or transcatheter procedure (e.g., transvascular, transfemoral, transeptal, etc.) to access the heart and native valve without requiring a full sternotomy.

Implementations disclosed herein can include an anchor, including anchor arms having a curved double hook shape. The anchors can be utilized in an anchoring system that can be dispensed with an anchor dispensing apparatus or can be utilized with another dispensing apparatus.

In some applications herein, an anchoring system is disclosed including a plurality of anchors each configured to anchor to tissue of a patient's body.

The anchoring system can include one or more tethers or other contraction members (e.g., wires, lines, tethers, ropes, ribbons, contraction wires, contraction lines, tensioning wires, tensioning lines, etc.) configured to couple the plurality of anchors to each other and to cinch the plurality of anchors.

The anchoring system can include an anchor dispensing apparatus having a central portion and including a plurality of dispenser arms circumferentially spaced from each other and each configured to extend radially outward from the central portion to a distal end of the respective dispenser arm, each dispenser arm configured to dispense at least one of the plurality of anchors to the tissue of the patient's body.

In some applications herein, a method is disclosed including positioning an anchor dispensing apparatus proximate a patient's native heart valve, the anchor dispensing apparatus having a central portion and including a plurality of dispenser arms circumferentially spaced from each other and extending radially outward from the central portion to a distal end of the respective dispenser arm.

The method can include dispensing a plurality of anchors from the plurality of dispenser arms to tissue surrounding the patient's native heart valve.

The method can include tensioning one or more tethers or other contraction members (e.g., wires, lines, tethers, ropes, ribbons, contraction wires, contraction lines, tensioning wires, tensioning lines, etc.) coupled to the plurality of anchors to cinch the plurality of anchors.

The above method(s) and steps can be performed on a living animal or on a simulation, such as on a cadaver, cadaver heart, simulator (e.g., with the body parts, heart, tissue, etc. being simulated), etc.

### BRIEF DESCRIPTION OF THE DRAWINGS

Features and advantages of the systems, apparatuses, and methods as disclosed herein will become appreciated as the same become better understood with reference to the specification, claims, and appended drawings wherein:

FIG. 1 illustrates a perspective view of an anchor according to an application of the present disclosure.

FIG. 2 illustrates a front view of the anchor shown in FIG. 1 in an undeployed or linearized configuration and positioned within a dispenser arm according to an application of the present disclosure.

FIG. 3 illustrates a perspective view of the anchor shown in FIG. 1 with a spacer body rotated from the position shown in FIG. 1.

FIG. 4 illustrates a side view of the anchor shown in FIG. 3, with a spacer body shown in cross section.

FIG. 5 illustrates a side schematic view of a plurality of anchors embedded within tissue.

FIG. 6 illustrates a side schematic view of the plurality of anchors shown in FIG. 5 cinched, tensioned, and/or contracted.

FIG. 7 illustrates a side cross sectional view of a tensioning and cutting mechanism according to an application of the present disclosure.

FIG. 8 illustrates a side cross sectional view of operation of the cutting mechanism shown in FIG. 7.

FIG. 9 illustrates a side cross sectional view of the tensioning and cutting mechanism shown in FIG. 7 being withdrawn from a lock.

FIG. 10 illustrates a side view of an anchor dispensing apparatus, with a plurality of shafts shown in cross section.

FIG. 11 illustrates a rear view of the anchor dispensing apparatus shown in FIG. 10.

FIG. 12 illustrates a distal perspective view of a portion of the anchor dispensing apparatus shown in FIG. 10.

FIG. 13 illustrates a proximal perspective view of a portion of the anchor dispensing apparatus shown in FIG. 10.

FIG. 14 illustrates a side perspective view of the anchor dispensing apparatus shown in FIG. 10, with dispenser arms extending radially outward.

FIG. 15 illustrates a side perspective view of the anchor dispensing apparatus shown in FIG. 10, with dispenser arms extending further radially outward than shown in FIG. 14.

FIG. 16 illustrates a side cross sectional view of a dispenser arm, having an anchor and a push body positioned therein according to an application of the present disclosure.

FIG. 17 illustrates a side cross sectional view of a dispenser arm, having an anchor and a push body slid from the position shown in FIG. 16 according to an application of the present disclosure.

FIG. 18 illustrates a side perspective view of the anchor dispensing apparatus shown in FIG. 10, with dispenser arms extending radially outward and having anchors positioned therein.

FIG. 19 illustrates a side perspective view of the anchor dispensing apparatus shown in FIG. 10, with dispenser arms extending radially outward and having anchors dispensed from the dispenser arms.

FIG. 20 illustrates a side schematic view of a dispenser arm contacting a wall.

FIG. 21 illustrates a schematic view of an anchor dispensing apparatus entering a patient's body according to an application of the present disclosure.

FIG. 22 illustrates a top schematic view of a left atrium and mitral valve, with an anchor dispensing apparatus positioned within the left atrium.

FIG. 23 illustrates a top schematic view of the left atrium and mitral valve shown in FIG. 22, with the dispenser arms of the anchor dispensing apparatus extending radially outward.

FIG. 24 illustrates a top schematic view of the left atrium and mitral valve shown in FIG. 22, with anchors dispensed around the mitral valve.

FIG. 25 illustrates a top schematic view of the left atrium and mitral valve shown in FIG. 22, with anchors cinched from the position shown in FIG. 24.

FIG. 26 illustrates a side view of an anchor, contraction member, and spacer body according an application of the present disclosure.

### DETAILED DESCRIPTION

Various aspects of the present disclosure generally relate to systems, apparatuses, and methods for medical treatment and/or treating heart conditions, including, by way of example, valve incompetencies (including valve regurgitation, which may include mitral valve regurgitation or tricuspid valve regurgitation). The systems, apparatuses, and methods can be adapted for percutaneous or transcatheter medical treatments without requiring full, open heart surgery. In some applications however, more invasive methods of delivery can be utilized. The systems, apparatus, and methods can be utilized to repair a heart valve by reshaping a heart valve annulus, including a mitral or tricuspid valve annulus, among other uses for other heart structures or other structures of the body.

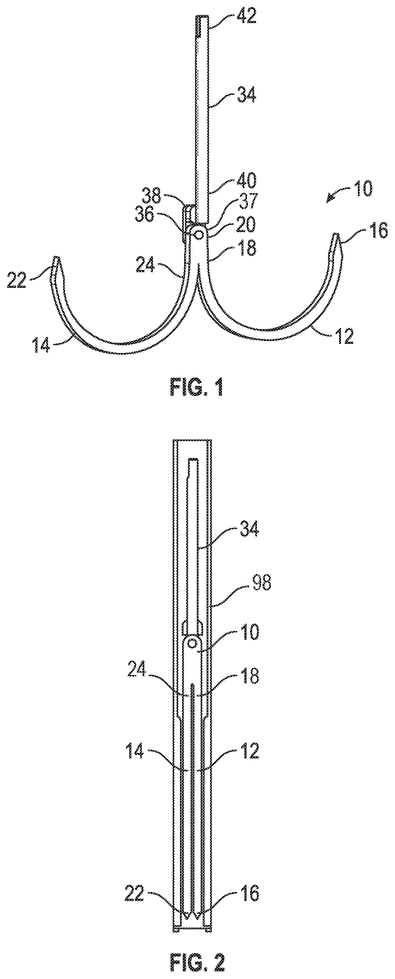

FIG. 1 illustrates an application of an anchor 10 according to an application of the present disclosure. The anchor 10 can include a first anchor arm 12 and a second anchor arm 14. The first anchor arm 12 can include a distal end including a puncturing tip 16 and can extend to a proximal portion 18 that is positioned at a central portion 20 of the anchor 10. Similarly, the second anchor arm 14 can include a puncturing tip 22 and can extend to a proximal portion 24 that is positioned at the central portion 20 of the anchor 10.

The central portion 20 of the anchor 10 can comprise a juncture that joins the proximal portions 18, 24 of the first anchor arm 12 and second anchor arm 14 together. The central portion 20 can comprise a linear body that can extend along an axis 39 (marked in FIG. 4). The anchor arms 12, 14 can be coupled together at the central portion 20 of the anchor 10 and each can extend outward from the central portion 20 to the tip 16, 22 of the respective anchor arm 12, 14. The anchor 10 can be configured to anchor to tissue of a patient's body.

The anchor 10 is shown in FIG. 1 in a deployed (or expanded) configuration, in which the anchor 10 has an expanded shape. Each anchor arm 12, 14 can form a curved hook, with the curved hooks extending outward from each other and from the central portion 20 of the anchor 10. Each anchor arm 12, 14 can extend away from the other arm in the same plane (co-planar) and outward from the central portion 20 of the anchor 10 to the tips 16, 22 of the respective anchor arm 12, 14 in opposite directions. Each anchor arm 12, 14 can have a convex curvature that curves in a distal direction. Each anchor arm 12, 14 can have a “U” shape or other shape as desired. The anchor 10 can have a double curved hook configuration.

The anchor arms 12, 14 can be shaped to extend outward symmetrically from the central portion 20 and can have the same shape as each other. In some applications, the shape of the anchor arms 12, 14 in the deployed configuration can differ from each other.

The anchor arms 12, 14 can be shaped to have the same width from the central portion 20 of the anchor 10 to the respective puncturing tip 16, 22 of each anchor arm 12, 14. The widths 26, 28 of each anchor arm 12, 14 are marked in FIG. 4. Each width 26, 28 can be set based on a desired width of securement into tissue for the respective anchor arm 12, 14. For example, the widths 26, 28 can be set to be between 5 millimeters and 10 millimeters in some applications, or can be about 8 millimeters in some applications, or can be greater or lesser as desired. Further, the depth 30, 32 of each anchor arm 12, 14 from the respective puncturing tip 16, 22 (marked in FIG. 4) can be set to a desired amount as well. For example, the depth 30, 32 can be between 3 and 6 millimeters and can be about 4 or 5 millimeters in some applications, or a greater or lesser amount. The radius of curvature of each anchor arm 12, 14 can be the same, and can be set to a desired amount. The radius of curvature for example, can between 5 millimeters and 10 millimeters, and can be about 8 millimeters in some applications, or can be a greater or lesser amount. A thickness 43 of the arms 12, 14 can be between 0.3 and 0.10 millimeters in some applications, and can be between about 0.5 to 0.7 millimeters in some applications, or can be a greater or lesser amount. The configurations and dimensions of the anchor arms 12, 14 can be varied in some applications as desired.

Referring back to FIG. 1, the anchor arms 12, 14 can be flat, and can be formed by being stamped or otherwise cut (e.g., laser cut) from a flat piece of material. The anchor arms 12, 14 of the anchor 10 can accordingly retain the flat shape of the material from which the anchor arms 12, 14 are formed. A thickness of the anchor arms 12, 14 can be between 0.2 and 0.5 millimeters, and can be about 0.4 millimeters in some applications, or can be a greater or lesser amount.

Each puncturing tip 16, 22 can be shaped as a sharpened tip configured to puncture tissue or other material as desired. The puncturing tips 16, 22 and anchor arms 12, 14 can be configured to penetrate and anchor to tissue, although in some applications the anchors 10 can anchor to other materials as well.

Each anchor arm 12, 14 can be biased to the deployed or expanded configuration shown in FIG. 1. In some applications, each anchor arm 12, 14 can be configured to move from an undeployed (or unexpanded, linearized, or straightened) configuration, to the deployed or expanded configuration. Each anchor arm 12, 14 for example, can be configured to deflect from a linearized shape in the undeployed configuration outward to the expanded shape shown in FIG. 1.

FIG. 2, for example, illustrates the anchor arms 12, 14 in the undeployed (or unexpanded, linearized, or straightened) configuration. The anchor 10 is shown positioned within a channel of a dispenser arm 98 according to an application of the present disclosure, with the dispenser arm 98 retaining the anchor arms 12, 14 in the undeployed configuration. The anchor arms 12, 14 have a linearized shape and extend axially relative to each other and are positioned adjacent to each other. The puncturing tips 16, 22 both extend distally and along the axis of the anchor 10 and are positioned adjacent to each other. The portion of the anchor arms 12, 14 between the tips 16, 22 and the proximal portions 18, 24 further extend along the axis of the anchor 10 and axially relative to each other. The axes of the anchor arms 12, 14 are parallel with each other. The anchor 10 is configured such that a distal force applied to the anchor 10 causes both puncturing tips 16, 22 to extend in the same distal direction and can thus puncture tissue or other material as desired in the same direction. The bias of the anchor arms 12, 14 to the deployed or expanded configuration can cause the anchor arms 12, 14 to deflect outward upon puncturing the tissue and other material, and thus expanding within the tissue or other material. The anchor arms 12, 14 can then form the double curved hook shape shown in FIG. 1 within the tissue or other material.

In some applications, the anchor 10 and anchor arms 12, 14 can be self-expanding and configured to self-expand from the undeployed configuration shown in FIG. 2 to the deployed configuration shown in FIG. 1. The anchor 10 and anchor arms 12, 14 can be formed in the configuration shown in FIG. 1 and biased towards such a shape. In some applications, the anchor 10 and anchor arms 12, 14 can be made of a shape memory material configured to expand to the deployed configuration shown in FIG. 1. For example, the anchor 10 and anchor arms 12, 14 can be made of a nitinol material or other form of shape memory material. In other applications, the anchor 10 and anchor arms 12, 14 can be constructed of another form of material (e.g., a spring biased material) that can be configured to deflect into the deployed configuration shown in FIG. 1.

In some applications, the anchor arms 12, 14 can be configured to retract back to the undeployed (or unexpanded, linearized, or straightened) configuration shown in FIG. 2. For example, the anchor 10 can be retracted back into the dispenser arm shown in FIG. 2 and can be linearized after being partially dispensed or the like from the dispenser arm 98.

Other configurations of anchors and anchor arms can be utilized according to various applications herein. For example, a greater number of anchor arms (e.g., at least three, four, or a greater number) can be utilized for an anchor according to various applications herein. At least two anchor arms can be utilized in some applications. Further, the shape of the anchor arms can be varied in some applications to angled shapes and other forms of curved shapes. The shape of the anchor arms can lack symmetry in some applications. As such, the configuration of the anchor utilized in some applications herein can be varied as desired.

Referring back to FIG. 1, a spacer body 34 can be coupled to the anchor 10. The spacer body 34 can be pivotally coupled to the anchor 10, with the anchor 10 including a pivot 36 that the spacer body 34 is configured to pivot about. The pivot 36 can be positioned at the central portion 20 of the anchor 10. The spacer body 34 can include a coupling body 38 that pivotally couples to the pivot 36 and can be configured to position the spacer body 34 offset from the plane that the anchor 10 extends in. As shown in FIG. 1, the coupling body 38 can extend proximally from the proximal end 37 of the anchor 10 and can form a plateau that an end 40 of the spacer body 34 is positioned upon, in a plane offset from the anchor 10.

In some applications, the spacer body 34 can comprise a tube having a first end 42 (or proximal end), a second end 40 (or distal end), and a length 48 therebetween (as marked in FIG. 4). The first end 42 and second end 40 can each include openings leading to an interior channel 44 (marked in FIG. 4) configured for one or more contraction members 46 (e.g., wires, lines, tethers, ropes, ribbons, contraction wires, contraction lines, tensioning wires, tensioning lines, etc.) to pass through. The contraction members 46 may be configured as tethers or other forms of contraction members 46 as desired.

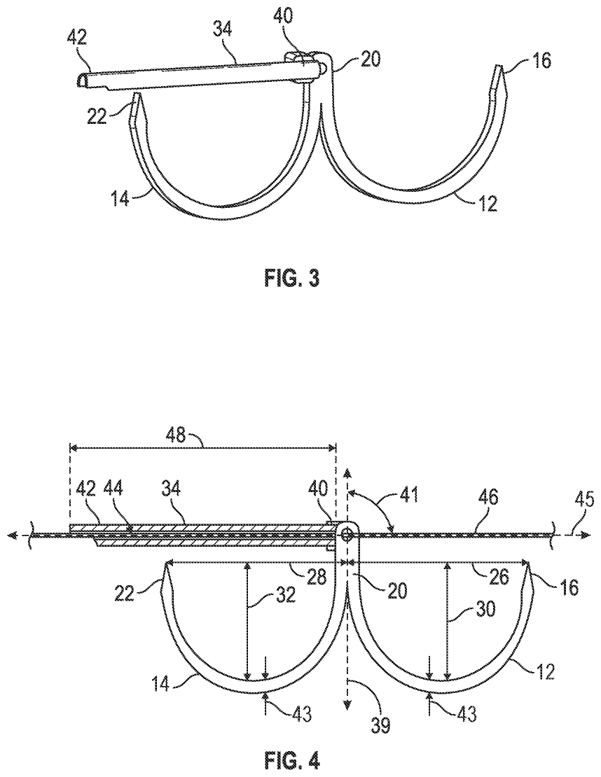

The spacer body 34 can be configured to pivot about the pivot 36 between a vertical or upright configuration in which the spacer body 34 extends axially with the central portion 20 of the anchor 10 as shown in FIG. 1, and a horizontal or rotated configuration in which the spacer body 34 extends transverse to the axis 39 of the central portion 20. A horizontal or rotated configuration is shown in FIG. 3. In the horizontal or rotated configuration, the spacer body 34 can extend at an angle 41 with respect to the axis 39 of the central portion 20 as shown in FIG. 4 that can be at ninety degrees or another amount as desired. The spacer body 34 may extend along an axis 45 (marked in FIG. 4) that extends transverse or perpendicular to the axis 39 of the central portion 20. The spacer body 34 can cover the width 28 of one of the anchor arms 14 and can be positioned closer to one of the tips 22 as shown in FIG. 4 in the horizontal or rotated configuration.

The spacer body 34 can be positioned in the upright configuration, extending axially with the anchor arms 12, 14 in the undeployed configuration as shown in FIG. 2. The spacer body 34 can extend axially relative to the anchor 10 when the anchor 10 is in an undeployed configuration. The spacer body 34 can extend axially with the anchor arms 12, 14 to allow for ease of deployment from the dispenser arm 98 shown in FIG. 2. Upon the anchor 10 and spacer body 34 being deployed from the dispenser arm, the spacer body 34 can then rotate to the horizontal or rotated configuration, as shown in FIG. 3.

The spacer body 34 can be configured to space adjacent anchors 10 from each other upon cinching of the anchors 10. The spacer body 34 can be configured to extend over or otherwise engage one or more contraction members 46 (e.g., wires, lines, tethers, ropes, ribbons, contraction wires, contraction lines, tensioning wires, tensioning lines, etc.). Referring to FIG. 4, a contraction member 46 can pass through the channel 44 of the anchor 10 and can be coupled to adjacent anchors. As the contraction member 46 is cinched, a first end 42 of the spacer body 34 can be drawn towards and contact a second end 40 of an adjacent spacer body 34. Similarly, the second end 40 of the spacer body 34 can contact the first end 42 of an adjacent spacer body 34. As such, the length 48 of the spacer body 34 can determine a distance between adjacent anchors 10 as the contraction member 46 is cinched. The offset of the spacer body 34 from the anchor 10 can allow the second end 40 of the spacer body 34 to directly contact the first end 42 of an adjacent spacer body 34. The length 48 can be set as desired. For example, in some applications the length can be between 5 millimeters and 10 millimeters, or a greater or lesser amount. In some applications, the length can be about 7 millimeters. Various spacer bodies used in an anchoring system can have differing lengths. For example, a length of one spacer body can be about 7 millimeters and a length of another spacer body can be about 9 millimeters. Various lengths can be utilized as desired.

As shown in FIG. 4, the first end 42 of the spacer body 34 can be configured to protrude from the puncturing tip 22 of the anchor 10. As such, space can be provided for the anchors 10 to be cinched together, with the spacer bodies 34 contacting each other and providing a spacing between the adjacent puncturing tips 16, 22 of the anchors 10. In other applications, other configurations of spacer bodies 34 can be utilized.

The one or more contraction members 46 (e.g., wires, lines, tethers, ropes, ribbons, contraction wires, contraction lines, tensioning wires, tensioning lines, etc.) can be configured to couple the plurality of anchors 10 to each other and to cinch the plurality of anchors 10. The one or more contraction members 46 can be configured to pass through the channel 44 of a spacer body 34 as desired. A contraction member 46 can be configured as a wire, line, or a cord, or other form of contraction member as desired. A contraction member 46 can be configured to be flexible, yet sturdy enough to resist the tension force applied to the contraction member 46. In some applications, a single contraction member or can be utilized or multiple contraction members can be utilized as desired.

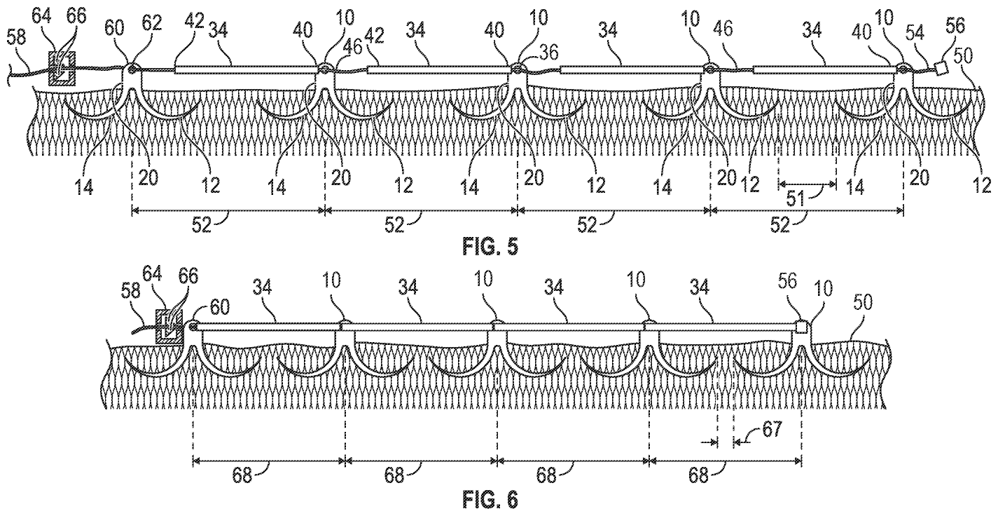

FIG. 5 illustrates an exemplary view of a plurality of the anchors 10 dispensed within tissue 50. The anchors 10 can be dispensed adjacent to each other and can have penetrated the tissue 50 in the undeployed or linearized configuration shown in FIG. 2. The anchors 10 can then have expanded to the deployed configuration shown in FIG. 1 within the tissue 50. The anchors 10 can self-expand into the deployed configuration shown in FIG. 1. The anchor arms 12, 14 can be entirely covered by the tissue 50, with the central portion 20 of the anchor 10 positioned above the surface of the tissue 50. As such, the pivots 36 and spacer bodies 34 can be positioned above the surface of the tissue 50.

The anchors 10 can be dispensed within the tissue 50 with a spacing 52 between the anchors 10. The spacing 52 can be between the central portions 20 of the anchors 10. Spacing 51 can be provided between adjacent puncturing tips 16, 22 of the anchors 10. In some applications, the spacing can be set such that adjacent puncturing tips overlap each other, or, as shown in FIG. 5, can be set with the tips 16, 22 of adjacent anchors 10 spaced from each other at a spacing 51.

The spacing 52 can be set as desired. For example, in some applications the spacing can be between 10 millimeters and 20 millimeters as desired, or a greater or lesser amount. In some applications, the spacing can be about 15 millimeters apart. The spacing can be set to provide a desired contraction of the tissue to which the anchors are dispensed in some applications.

The anchors 10 can be dispensed within the tissue 50 with the spacer bodies 34 positioned in the upright configuration as shown in FIG. 1. One or more contraction members 46 can pass through the interior channels of the spacer bodies 34 and couple the anchors 10 to each other. The anchors 10 accordingly can be dispensed with a contraction member 46 coupling all the anchors 10 and passing through the spacer bodies 34. Such a configuration, for example, is shown in FIG. 19. A distal end 54 of the contraction member 46 can couple to a stopper 56 that can be sized larger than the channel of the adjacent spacer body 34 and configured to contact a second end 40 of the adjacent spacer body 34. As such, the stopper 56 can prevent the end 54 of the contraction member 46 from passing through the adjacent spacer body 34 and can resist a cinching force applied to the contraction member 46 from an opposite proximal portion 58 of the contraction member 46.

The spacer bodies 34 can pivot to the horizontal or rotated configuration shown in FIG. 5, which can occur by partially cinching the contraction member 46, causing the spacer bodies 34 to pivot horizontally with respect to the surface of the tissue 50. A proximal-most anchor 60 can be configured similarly as the remaining anchors 10. The proximal-most anchor 60, however, can include an aperture 62 for the contraction member 46 to pass through, and may not need a spacer body 34, as the adjacent anchor 10 can include the spacer body 34 for spacing a distance from the anchor 60.

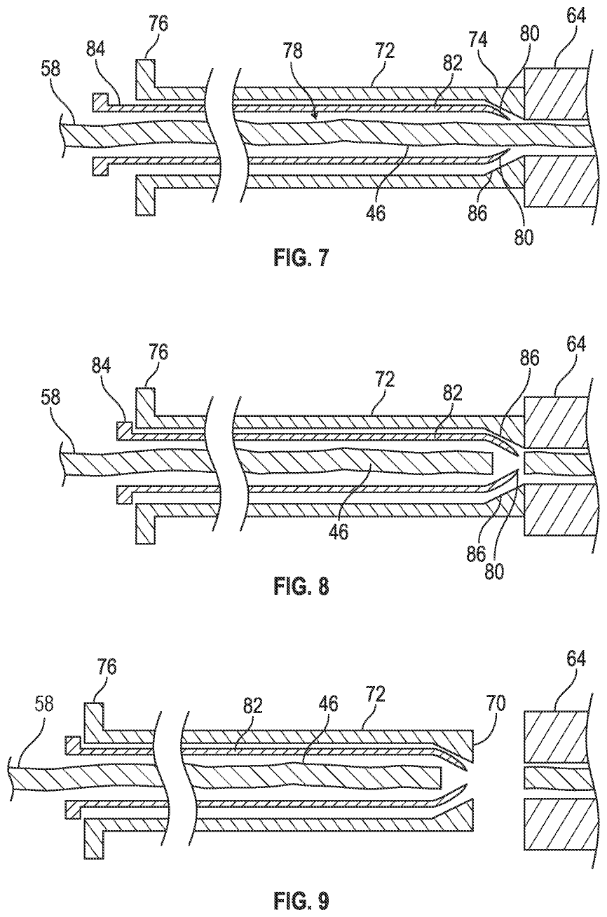

The proximal portion 58 of the contraction member 46 can be coupled to a tensioning mechanism (as can be shown in FIG. 7) that can be configured to tension the one or more contraction members 46 (e.g., wires, lines, tethers, ropes, ribbons, contraction wires, contraction lines, tensioning wires, tensioning lines, etc.). The anchors 10 accordingly can comprise a distal anchor (the right-most anchor shown in FIG. 5), a proximal anchor 60, and one or more intermediate anchors 10 (between the distal anchor and the proximal anchor 60 shown in FIG. 5). The contraction member 46 can be tensioned at the proximal anchor 60.

A lock 64 can be positioned on the proximal portion 58 of the contraction member 46 and proximal of the anchor 60. The lock 64 can be configured to lock the one or more contraction members 46 in a tensioned configuration. The lock 64 can be configured as a ratcheting lock, that can allow movement of the lock 64 in a distal direction and can prevent movement of the lock 64 in a proximal direction. As such, the lock 64 can include locking surfaces 66 configured to lock against the proximal portion 58 of the contraction member 46 to prevent the contraction member 46 from being pulled distally away from the lock 64. A variety of different forms of locks, including ratcheting locks, can be utilized in some applications. In some applications, a user operated or set lock, or resettable lock can be utilized to release and set the lock as desired. Such a feature can be beneficial if over-tensioning of the contraction member is determined during a procedure and the lock must be reset, or there is another need for releasing and/or re-setting the lock.

Upon the anchors 10 being deployed in the desired positions, the anchors 10 can be cinched by the contraction member 46 being cinched. A tensioning mechanism, for example as shown in FIG. 7 can be utilized to cinch the contraction member 46. The contraction member 46 can be pulled proximally to cinch the contraction member 46 which can cause the stopper 56 to abut the end 40 of the adjacent spacer body 34 and apply a proximal force to the anchor 10 and spacer body 34. The spacer body 34 can then be moved proximally towards an adjacent spacer body 34, which can contact the end 40 of another adjacent spacer body 34. Such a motion can reduce the spacing 52 between the anchors 10. The spacing between the anchors 10 can continue to be reduced, thus cinching the anchors 10 together.

A resistive force can be applied to the lock 64 and the anchor 60 by the tensioning mechanism. The resistive force can be in a distal direction to prevent the anchor 60 from moving proximally upon the contraction member 46 being cinched. Such a resistive force can further allow the spacing 52 between the anchors 10 to be decreased.

FIG. 6, for example, illustrates the anchors 10 having been cinched. The spacing 68 between the anchors 10 is reduced from the spacing 52 shown in FIG. 5. Further, the spacing 67 between the tips of the anchor arms is reduced from the spacing 51 shown in FIG. 6. The spacer bodies 34 are in contact with each other and define a spacing between the anchors 10. The lock 64 can be pressed against the anchor 60 and locked to the proximal portion 58 of the contraction member 46 to hold the contraction member 46 in a tensioned configuration. The locking surfaces 66 can hold against the contraction member 46. The proximal portion 58 of the contraction member 46 can be cut with a cutting mechanism to reduce the amount of contraction member 46 extending from the proximal anchor 60 and proximally from the lock 64.

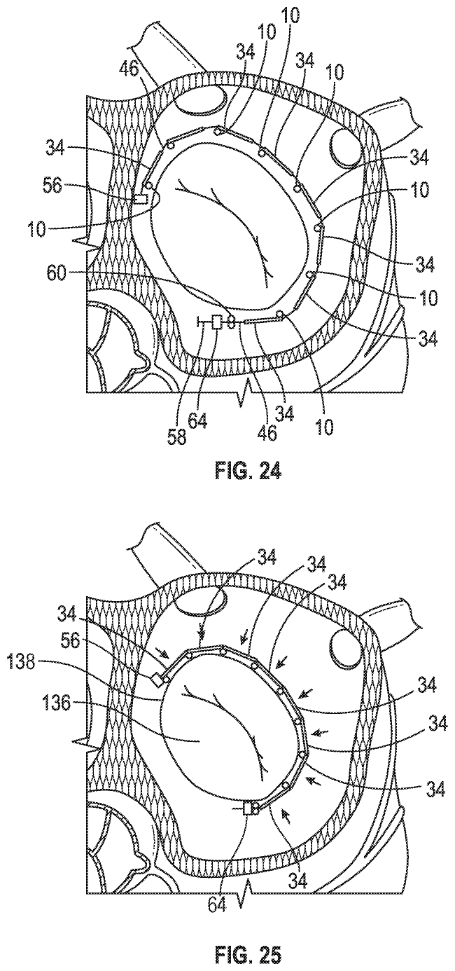

The configuration of the anchors 10 shown in FIGS. 5 and 6 can beneficially provide an improved anchoring of the anchors 10 to the tissue. Further, the double curved hook configuration of the anchor arms 12, 14 can improve the grip of the anchors 10 to the tissue and can reduce the total number of anchors 10 that may be needed to contract the underlying tissue 50. The use of the anchors 10 and the anchoring system can be utilized in a variety of manners, including reducing a size of a valve annulus as shown for example, in FIGS. 24 and 25. The anchors 10 can each be configured to anchor to tissue within a patient's heart and surrounding a native valve. The anchors 10 can be positioned in a curve about the native heart valve and used to reduce the size of the annulus. The valve can be a mitral valve or tricuspid valve or other valve in some applications. Other uses can be provided, including general retraction or compression of tissue. Variations in the use of the anchors 10 and the anchoring system can be utilized.

The anchors 10 disclosed herein can be utilized solely, without the other components disclosed herein. Further, the anchoring system including anchors 10, spacer bodies, and one or more contraction members, can be utilized solely, without the other components disclosed herein. For example, the anchors 10 can be utilized without use of the anchor dispensing apparatus 88 disclosed herein.

FIG. 7 illustrates a side cross sectional view of a tensioning mechanism and cutting mechanism that can be utilized according to various applications herein. The tensioning mechanism can include a pressing surface 70 (marked in FIG. 9) that can be configured to apply a force to a portion of the anchoring system, particularly the lock 64 or anchor 60 shown in FIG. 5. The pressing surface 70 can comprise a distal surface of the tensioning mechanism. The tensioning mechanism can include a shaft 72, and the pressing surface 70 can be a distal surface of the shaft 72. The shaft 72 can comprise the shaft of a catheter that is configured to be passed into a patient's body. The shaft 72 can have a distal end 74 including the pressing surface 70 and can have a proximal portion 76 that can be configured to be positioned outside of the patient's body during operation of the tensioning mechanism. The proximal portion 76 can be accessible by a user and configured to be moved during a treatment procedure. The shaft 72 can be configured to transmit a pressing force from the proximal portion 76 of the shaft 72 to the distal end 74 and pressing surface 70 of the shaft 72.

The shaft 72 in some applications can comprise a sheath that extends around a channel 78. The channel 78 can be configured for the contraction member 46 to pass through, with the proximal portion 58 of the contraction member 46 being accessible at the proximal portion 76 of the shaft 72. The proximal portion 58 can be accessible by a user and configured to be moved (e.g., pulled distally) during a treatment procedure from outside the patient's body.

In some applications, the cutting mechanism can be coupled to the tensioning mechanism. The cutting mechanism can be configured to cut the one or more contraction members 46 in a tensioned configuration. For example, as shown in FIG. 7, the cutting mechanism can include cutting surfaces 80 that are positioned at a distal end of a cutter 82. The cutter 82, for example, can comprise a shaft that extends within the channel 78 and has a proximal portion 84 that can be accessible to be operated by a user. The proximal portion 84 can be accessible by a user and configured to be moved during a treatment procedure.

The cutter 82 can be configured to deflect against angled surfaces 86 of the shaft 72, which can deflect the cutting surfaces 80 against the contraction member 46 to cut the contraction member 46 upon the cutter 82 being advanced distally. In some applications, the cutting mechanism can have another configuration, and may not be positioned within the shaft of the tensioning mechanism. In some applications, the cutting mechanism can be a separate device, including a shaft or catheter that is configured to cut the contraction member 46 in a tensioned configuration. The configuration of the cutter 82 can further be varied from the configuration shown in FIG. 7.

In operation, the tensioning mechanism can be advanced along the proximal portion 58 of the contraction member 46 in a distal direction. The tensioning mechanism can be advanced after the anchors 10 have been dispensed to a desired implantation site or can be in place during the dispensing of the anchors 10. The tensioning mechanism can be advanced with the pressing surface 70 pressing the lock 64 and advancing the lock 64 along the contraction member 46 toward the proximal anchor 60. The tensioning mechanism can be advanced distally until the lock 64 presses against the anchor 60.

The proximal portion 58 of the contraction member 46 can be accessible to a user and thus can be pulled proximally as the pressing surface 70 is advanced distally. For example, a user can grip the proximal portion 76 of the tensioning mechanism and advance the proximal portion 76 distally or can hold the proximal portion 76 in position as the contraction member 46 is pulled proximally. A tension measurement device, for example, can be utilized to determine whether a desired amount of tension is applied to the contraction member 46, and whether the anchors 10 as shown in FIG. 6 are cinched to a desired amount. Visualization of the anchors 10 via fluoroscopy and/or echocardiography, among other forms of visualization, can alternatively or additionally in combination with the tension measurement device, be utilized to determine whether the anchors 10 are cinched to a desired amount. The tension in the contraction member 46 can be increased until a desired amount of tension is provided. In an application in which the anchors 10 are deployed to a heart valve in a beating heart operation, measurement of the operation of the heart valve (e.g., flow and/or pressure gradient) can be performed to determine if desired tension is applied to the contraction member 46.

The lock 64 can be configured to resist movement of the contraction member 46 in a distal direction, and thus can lock the tension of the contraction member 46 upon the tension being set to a desired amount. With the tension set, the cutting mechanism can be utilized to cut the proximal portion 58 of the contraction member 46. The proximal portion 84 of the cutter 82, for example, can be advanced distally to cause the cutting surfaces 80 to slide against the angled surfaces 86 and cut the contraction member 46. FIG. 8, for example, illustrates the cutter 82 cutting the contraction member 46.

With the lock 64 maintaining the tension in the contraction member 46, and the proximal portion 58 of the contraction member 46 cut, the shaft 72 of the tensioning mechanism and the cutter 82 of the cutting mechanism, as well as the remaining proximal portion of the contraction member 46 can be retracted proximally and out of the patient's body. The lock 64 can remain in position with the anchors 10 cinched and the contraction member 46 locked in a tensioned configuration.

In some applications, other forms of tensioning mechanisms and cutting mechanisms can be utilized as desired. The tensioning mechanism and cutting mechanism can be separate from each other or combined as desired.

The tensioning mechanism and cutting mechanism disclosed herein can each be utilized solely, without the other components disclosed herein.

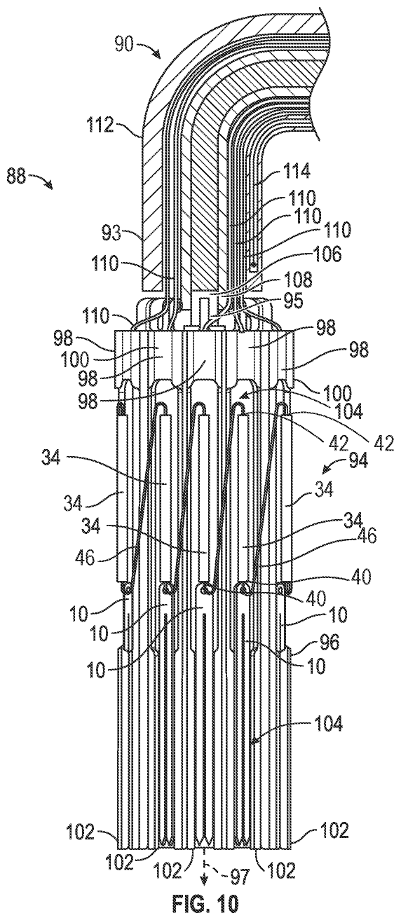

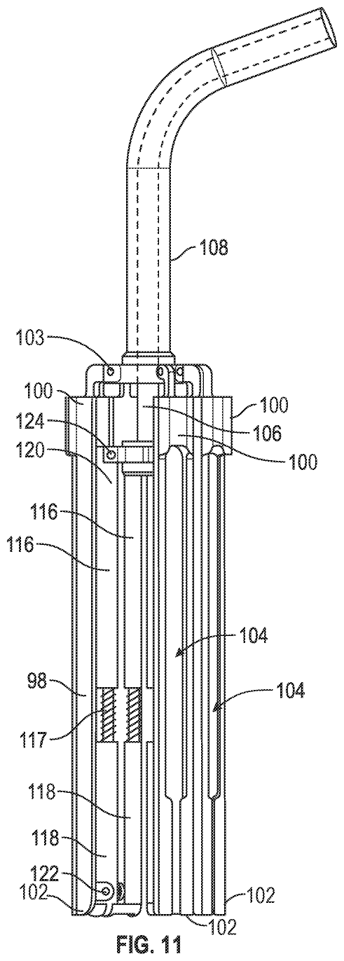

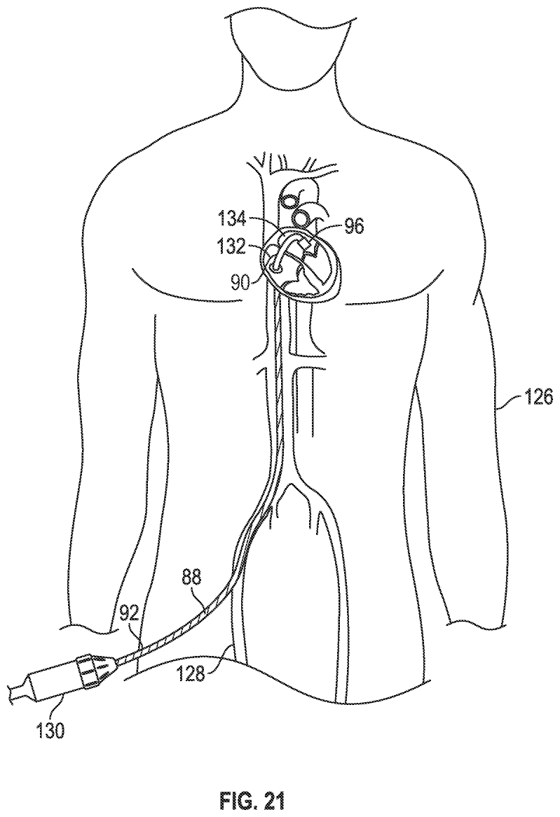

FIG. 10 illustrates an application of an anchor dispensing apparatus 88 that can be utilized according to applications herein. The anchor dispensing apparatus 88 can include an elongate shaft 90 having a proximal end 92 (marked in FIG. 21), and a distal end 93 (marked in FIG. 10). The anchor dispensing apparatus 88 can have a distal end 94 comprising a dispensing head 96 that is positioned at the distal end 93 of the elongate shaft 90. The dispensing head 96 can be configured to dispense a plurality of anchors 10 to a portion of the patient's body.

The dispensing head 96 of the anchor dispensing apparatus 88 can include a central portion 95 having a central axis 97. The dispensing head 96 can include a plurality of dispenser arms 98. Each dispenser arm 98 can be circumferentially spaced from each other. Each dispenser arm 98 can be positioned about the central axis 97 of the central portion 95.

Each dispenser arm 98 can have a proximal portion 100 having a proximal end, and a distal portion 102 including a distal end. Each dispenser arm 98 can be an elongate linear body and extend longitudinally along the central axis 97 from the proximal end to the distal end of the respective dispenser arm 98 in the undeployed (or unexpanded) configuration shown in FIG. 10. Each dispenser arm 98 can have an axis that extends parallel with an axis of each other dispenser arm 98, and parallel with the central axis 97, in the undeployed configuration shown in FIG. 10. Each dispenser arm 98 can extend distally from the elongate shaft 90 to the distal end of the respective dispenser arm 98 in the undeployed (or unexpanded) configuration shown in FIG. 10.

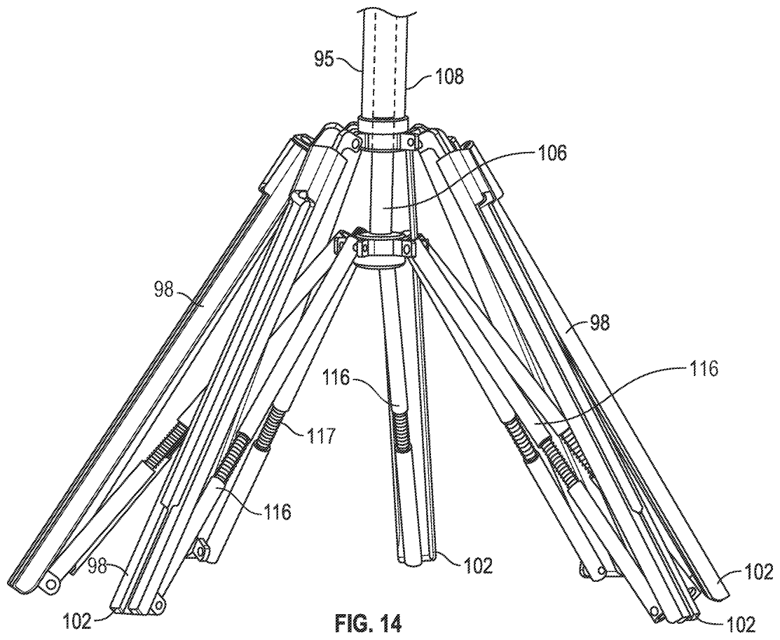

The proximal portion 100 of each dispenser arm 98 can be pivotally coupled to the central portion 95 of the anchor dispensing apparatus 88. A pivot 103 for each dispenser arm 98 (as marked in FIG. 13) can couple the proximal portion 100 to the central portion 95.

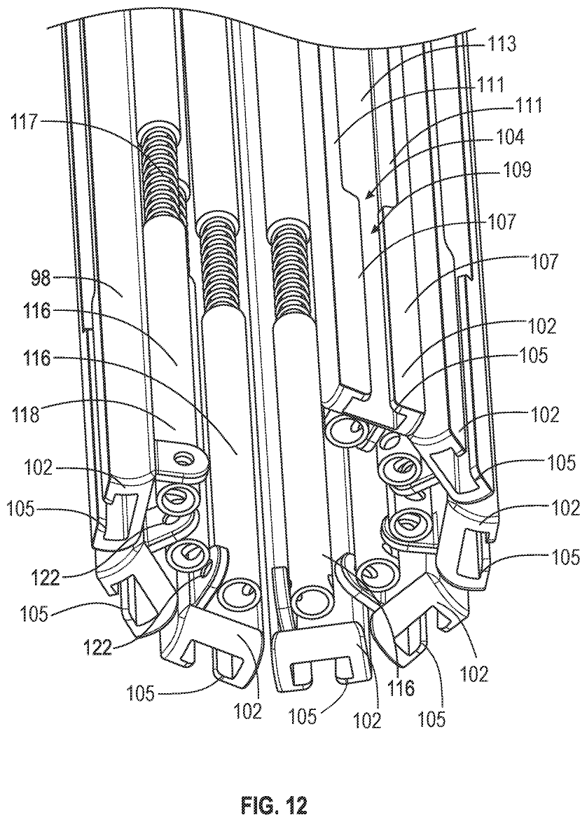

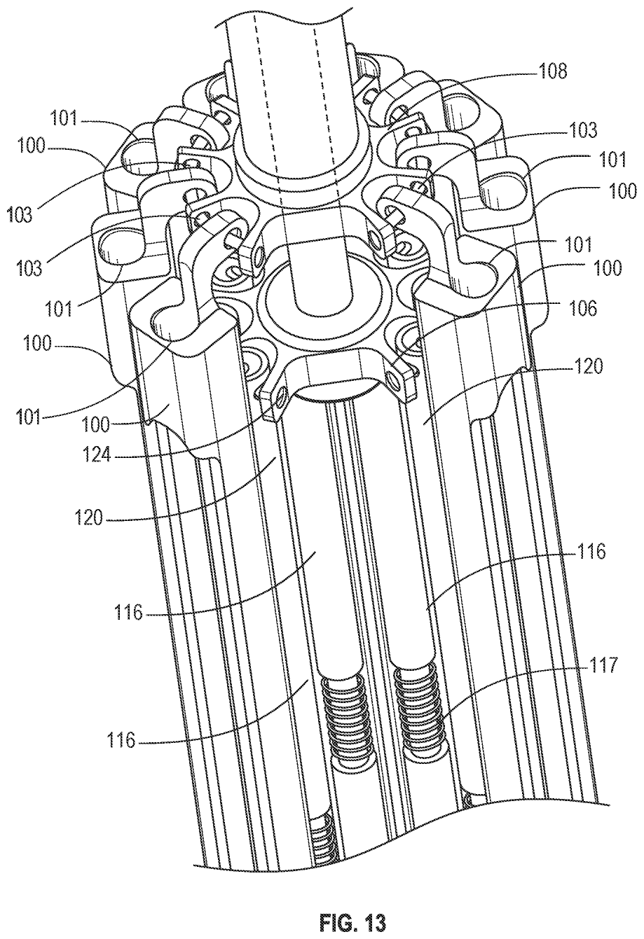

The distal portion 102 of each dispenser arm 98 can include a distal end that is configured for the anchor 10 to be dispensed from. Each distal end can include an opening 105 (marked in FIG. 12) for an anchor 10 to be dispensed from. Further, each proximal end can include an opening 101 (marked in FIG. 13) that is configured for a respective push body 110 to pass through to push an anchor 10 from the respective dispenser arm 98.

Each dispenser arm 98 can include a channel 104 for the anchor 10 to slide within. Further, the spacer body 34 can be coupled to the anchor 10 and can slide along with the anchor 10. The channel 104 can be configured to retain the anchor 10 and the spacer body 34. The channel 104 can be configured for the anchor 10 and the spacer body 34 to be passed through in an undeployed or linearized configuration, as shown in FIG. 2 for example. The anchor 10 and the spacer body 34 can be slid distally along the channel 104 to be dispensed from the distal end of the respective dispenser arm 98, upon which the anchor 10 can expand to the deployed or expanded configuration within the patient's tissue or other material.

Each dispenser arm 98 can be shaped to retain the anchor 10 and spacer body 34 within the channel 104 in the linearized configuration. Each dispenser arm 98 can be configured to dispense at least one of the plurality of anchors 10 to the tissue of the patient's body.

FIG. 12 for example, illustrates a close up view of a distal portion of a channel 104. The dispenser arm 98 can include retaining lips 107 extending over the channel 104, with a slot 109 extending between the lips 107 and along the channel 104 for the spacer body 34 to pass through. The channel 104 can be bounded by side walls 111 extending along the channel 104 and being raised from a base wall 113.

In some applications, each dispenser arm 98 can retain an anchor 10 in the undeployed or linearized configuration. The side walls 111 for example, can hold the anchor 10 in the undeployed or linearized configuration as shown in FIG. 10.

The configuration of the dispenser arm 98 and channel can be varied in some applications. For example, in some applications, the anchor 10 can be retained outside of the channel 104, yet can slide relative to the channel 104 and dispenser arm 98. Further, in some applications, the dispenser arm 98 can retain the anchor 10 at the distal end of the arm 98 and dispense the anchor 10 by delivering the anchor 10 from the distal end. For example, the anchor 10 can be pressed against a surface by the dispenser arm 98 for the anchor 10 to be dispensed. Various forms of dispensing by the dispenser arm 98 can be provided in some applications.

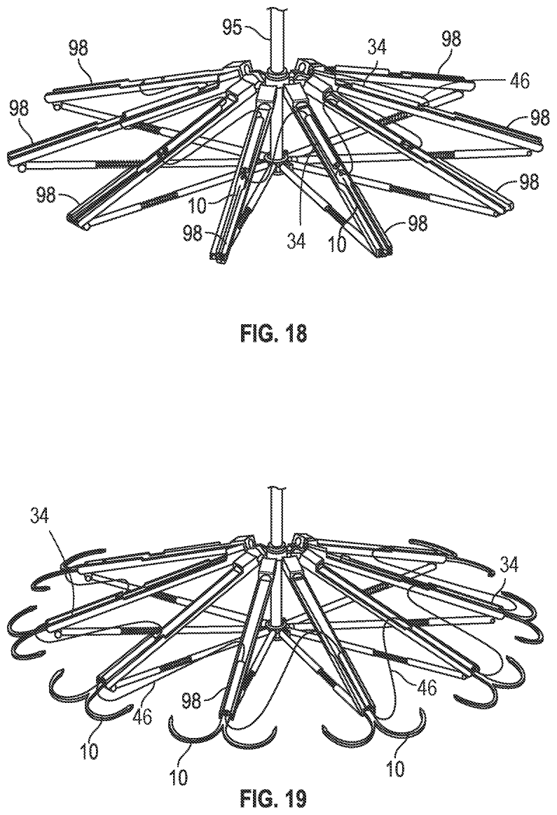

Referring to FIG. 10, one or more contraction members 46 (e.g., wires, lines, tethers, ropes, ribbons, contraction wires, contraction lines, tensioning wires, tensioning lines, etc.) can pass through the spacer bodies 34, passing in a zig-zag manner from a proximal end 42 of a spacer body 34, to a distal end 40 of an adjacent spacer body, then through the spacer body 34 to the proximal end 42, and then to a distal end 40 of an adjacent spacer body. The one or more contraction members 46 accordingly can extend around the central axis 97 and around the dispenser arms 98 and can be routed in a configuration for deployment. FIGS. 18 and 19, for example, illustrate the anchors 10 being dispensed with a contraction member 46 extending between the spacer bodies 34.

The central portion 95 of the anchor dispensing apparatus 88 can include a central support 106 and can include a slide body 108. The central support 106 is shown in partial cross section in FIG. 10, and the slide body 108 is shown in cross section in FIG. 10. Perspective views of applications of the central support 106 and slide body 108 are shown in FIG. 14, with the push bodies 110 and steerable shaft 112 removed from view.

The slide body 108 can be configured to slide relative to the central support 106. The proximal portions 100 of the dispenser arms 98 can be pivotally coupled to the slide body 108, and thus the proximal portions 100 can be pivotally coupled to the central support 106 via the coupling to the slide body 108. In some applications, for example, as shown in FIG. 10, the slide body 108 can comprise a shaft in the form of a sheath extending around the central support 106 or can comprise a ring or other form of body extending around the central support 106. The slide body 108 can extend along the length of the elongate shaft 90 of the anchor dispensing apparatus 88 and can have a proximal portion that is accessible outside of the patient's body. For example, the proximal portion can extend to the handle 130 shown in FIG. 21.

The central support 106 can comprise a shaft that extends within the slide body 108 and along the length of the elongate shaft 90 of the anchor dispensing apparatus 88. The central support 106 can have a proximal portion that is accessible outside of the patient's body. For example, the proximal portion can extend to the handle 130 shown in FIG. 21.

The anchor dispensing apparatus 88 can further include a dispensing mechanism configured to dispense the plurality of anchors 10 from the plurality of dispenser arms 98. The dispensing mechanism can include one or more push bodies 110 that can be configured to push the anchors 10 distally from the plurality of dispenser arms 98. The push bodies 110 can pass through the channel 104 of a respective dispenser arm 98. The push bodies 110 can push the anchor 10 out of the channel 104 in a distal direction. The push bodies 110 for example can comprise wires that pass through proximal openings 101 of the dispenser arms 98 (as shown in FIG. 13) and extend through the channel 104 in a distal direction. The wires can comprise a flexible material and can comprise a shape memory material such as nitinol or another form of shape memory material. In some applications, other materials can be utilized for the push bodies 110.

Proximal portions of the push bodies 110 can extend along the elongate shaft 90 and can be accessible at the proximal end of the elongate shaft 90 and outside of the patient's body. For example, the proximal portion can extend to the handle 130 shown in FIG. 21.

The push bodies 110 can be positioned around the slide body 108 as shown in FIG. 10 or can have another configuration as desired. A plurality of push bodies 110 can extend around the slide body 108 and along the length of the elongate shaft 90, with one push body 110 per dispenser arm 98 or another amount as desired. In some applications, the configuration and operation of the dispensing mechanism can be varied as desired, for example, other forms of dispensing such as rotatable drives or geared conveyors can be utilized to dispense the anchors. Electrical (or electromagnetic) dispensing can be utilized in some applications.

The anchor dispensing apparatus 88 can further include a steerable shaft 112 that can form an outer sheath of the elongate shaft 90. The outer sheath can extend over the push bodies 110, slide body 108, and central support 106 of the elongate shaft 90. The steerable shaft 112 can include a pull wire 114 or the like that can allow the steerable shaft 112 to be steered and maneuvered as desired. The steerable shaft 112 can allow the dispensing head 96 to be deflected in a single plane, or in some applications can be deflected in multiple planes, including at least two planes. The steerable shaft 112 can allow the dispensing head 96 to be oriented in a desired orientation relative to an implantation site, such as a native heart valve, including a native mitral or tricuspid valve. In some applications, the steerable shaft 112 can be excluded, and the dispensing head 96 can be positioned in a pre-set orientation or can be configured to passively orient into a position. In some applications, other forms of steerable shafts 112 can be utilized, such as shafts forming an interior shaft of the anchor dispensing apparatus 88, and other forms of steering mechanisms for the dispensing head 96.

The steerable shaft 112 can be operated to deflect the dispensing head 96 via a steering mechanism control at a housing such as the handle 130 shown in FIG. 21 for example.

An actuation mechanism can be utilized to cause each of the dispenser arms 98 to extend radially outward from the central portion 95 of the anchor dispensing apparatus 88 to a distal end of the respective dispenser arm 98. The actuation mechanism can be configured to cause the dispenser arms 98 to pivot from an undeployed (or unexpanded) configuration radially outward to a deployed (or expanded) configuration.

The actuation mechanism can include a shaft in the form of the central support 106, a shaft in the form of the slide body 108, and a plurality of support arms 116 (visible in FIG. 11). FIG. 11, for example, illustrates a rear perspective view of the anchor dispensing apparatus 88 with the anchors 10 and spacers 34 excluded from view, and the push bodies 110 and steerable shaft 112 excluded from view. The central support 106 is shown to comprise a shaft, with the slide body 108 comprising a sheath extending over the shaft.

The dispenser arms 98 are shown to not extend fully around the central axis 97, and a space can be provided where dispenser arms 98 are not positioned. Such positioning can accommodate the configuration of the anchors to be dispensed at the treatment site. In some applications, the dispenser arms 98 can extend fully around the central axis 97.

Referring to FIG. 11, the support arms 116 can each have a distal portion 118 including a distal end pivotally coupled to a respective one of the dispenser arms 98 and including a proximal portion 120 pivotally coupled to the central support 106. The distal portion 118 of the respective support arm 116 can be coupled to the distal end of the respective one of the plurality of dispenser arms 98. A pivot 122 can couple the distal portion 118 to a distal end of the respective dispenser arm 98, and a pivot 124 can couple the proximal portion 120 to the central support 106, for example to the distal end of the central support 106. The support arms 116 can be configured to pivot from an undeployed (or unexpanded) configuration radially outward to a deployed (or expanded) configuration.

Each support arm 116 can include a compressible body 117 that can be positioned along the length of the support arm 116. Each support arm 116 can be configured to be compressed in a direction towards the proximal portion 120 of the respective one of the plurality of support arms 116. The compressible body 117 for example, can comprise a spring or another form of compressible body that can allow the support arm 116 to be compressed. The compressible body 117 can further be resilient and biased to not be compressed, and thus can apply a force in a distal direction to the respective dispenser arm 98. The support arm 116 can provide a spring bias force radially outward against the respective one of the plurality of dispenser arms 98.

FIG. 11 illustrates an undeployed (or unexpanded or linearized) configuration of the anchor dispensing apparatus 88. The dispenser arms 98 can extend axially relative to each other in the undeployed configuration. Further, each support arm 116 can extend axially relative to each other and to the dispenser arms 98 in the undeployed configuration. As shown in FIG. 11, each dispenser arm 98 can overlay the support arm 116 and be positioned exterior of the support arm 116 when the anchor dispensing apparatus 88 is in an undeployed (or unexpanded or linearized) configuration.

FIG. 12 illustrates a distal perspective view of the distal ends of the dispenser arms 98 and the support arms 116. FIG. 13 illustrates a proximal perspective view of the proximal ends of the dispenser arms 98 and the support arms 116.

The actuation mechanism can operate by the central support 106 being slid distally relative to the slide body 108. The pivotal coupling of the dispenser arms 98 and the support arms 116 can cause the support arms 116 to pivot radially outward from the central portion 95 of the anchor dispensing apparatus 88 and thus causing the dispenser arms 98 to pivot and extend radially outward from the central portion 95 as well. FIG. 14, for example, illustrates the central support 106 being slid distally relative to the slide body 108. The support arms 116 and dispenser arms 98 extend radially outward from the central portion 95. The circumferential distance between the distal portions 102 of the dispenser arms 98 increases as the dispenser arms 98 are spread outward from each other.

The actuation mechanism can be controlled at the proximal portions of the central support 106 and slide body 108. For example, the proximal portions can be controlled to cause the desired movement at the distal portions of the central support 106 and slide body 108. A user, for example, can grasp and move the proximal portions to cause the relative movement at the distal portions.

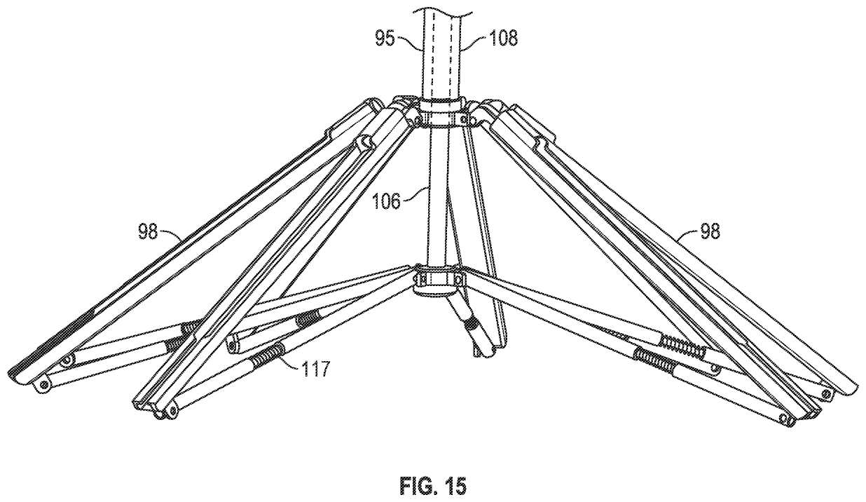

The actuation mechanism can continue to operate to extend the dispenser arms 98 outward to a desired radius from the central portion 95 of the anchor dispensing apparatus 88. FIG. 15, for example, illustrates the central support 106 continuing to slide relative to the slide body 108 distally, further extending the dispenser arms 98 radially outward. The diameter of the anchor dispensing apparatus 88 with the dispenser arms 98 in the deployed configuration is greater than the diameter of the anchor dispensing apparatus 88 with the dispenser arms 98 in the undeployed configuration (as shown in FIG. 11).

The distal ends of the dispenser arms 98 can be circumferentially spaced from each other, and can be spaced equally from each other, when the dispenser arms 98 extend radially outward from the central portion 95 in the deployed configuration. In some applications, other amounts of spacing between the distal ends of the dispenser arms 98 can be utilized, which can be based on the configuration of anchors 10 to be provided at the treatment site.

With the dispenser arms 98 extending radially outward from the central portion 95 to a desired amount, the anchors 10 can be dispensed from the dispenser arms 98. The dispenser arms 98 can then be retracted radially inward in a reverse operation for removal from the treatment site.

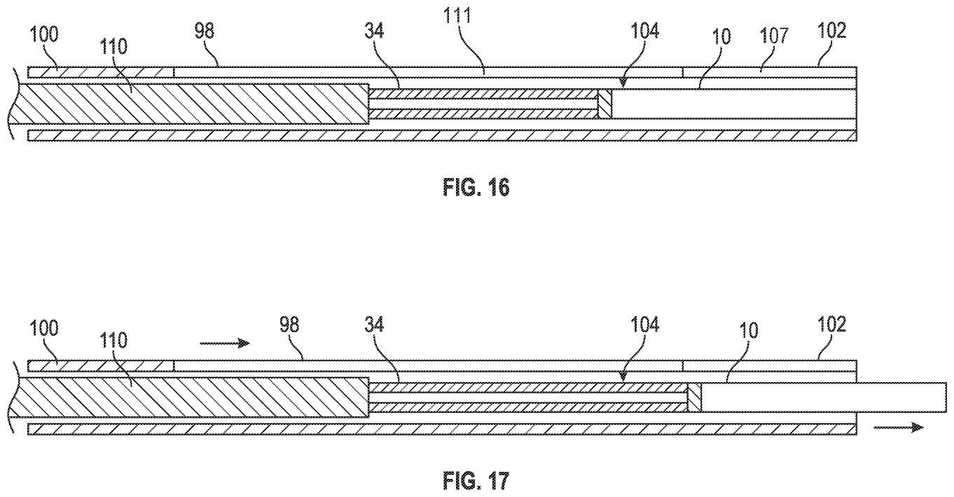

FIG. 16, for example, illustrates a cross sectional view of a channel 104 of a dispenser arm 98 along a center line of the channel 104, with an anchor 10 and spacer body 34 positioned therein.

The push body 110 can slide distally within the channel 104 to press against the spacer body 34 and anchor 10 to dispense the anchor 10 from the dispenser arm 98. In the implementation shown in FIG. 16, the spacer body 34 can be positioned in-line with the anchor 10, however, in some applications, the spacer body 34 can be positioned offset from the anchor 10 and the push body 110 can press directly against the anchor 10 to dispense the anchor 10 from the dispenser arm 98.

FIG. 17 illustrates the push body 110 pushing the anchor 10 from the dispenser arm 98. The anchor 10 can move to the expanded state upon being dispensed from the dispenser arm 98.

FIG. 18, for example, illustrates a perspective view of the dispenser arms 98 extending radially outward from the central portion 95, with the anchors 10 coupled thereto. The contraction member 46 is shown routed through the spacer bodies 34 from one end of the spacer body to another end of the adjacent spacer body 34. The contraction member 46 can be routed in a zig-zag manner such that the contraction member 46 is routed in position for the anchors 10 to be dispensed. In other applications, other configurations or routings of contraction members can be utilized.

FIG. 19 illustrates the anchors having been dispensed from the respective dispenser arms 98. Each anchor 10 can expand to the expanded configuration shown in FIG. 1 upon extending from the dispenser arms 98. The distal end of each dispenser arm 98 can be in contact with or can be proximate a surface of tissue or other material for the anchor 10 to penetrate into.

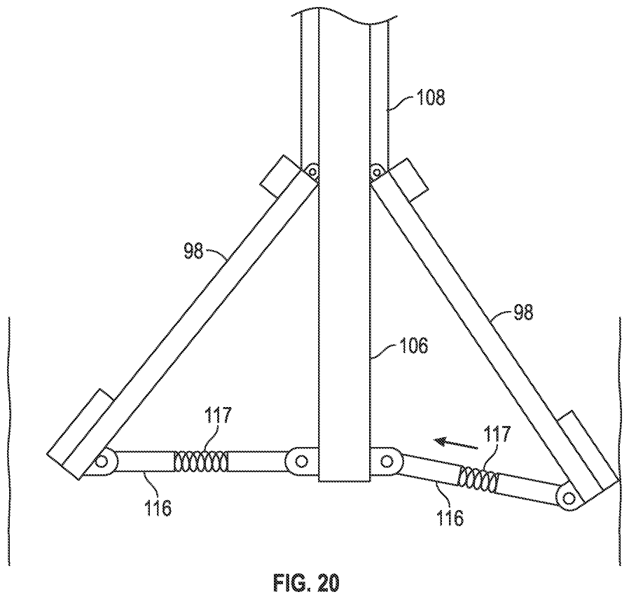

FIG. 20 illustrates a schematic view of a feature of the compressible body 117 of the support arms 116. As the dispenser arms 98 are expanded, the dispenser arm 98 can contact a surface, such as a surface of a patient's heart including an atrial wall or other surface. The compressible body 117 for the dispenser arm 98 that contacts the surface (as shown to the right in FIG. 20) can compress, thus allowing another dispenser arm 98 that has not contacted a surface (as shown to the left in FIG. 20) to continue to expand radially outward. As such, contact of a dispenser arm 98 against a surface may not impede further expansion of other dispenser arms 98.

The configuration of the anchor dispensing apparatus disclosed herein can be varied in some applications as desired. For example, in some applications a connecting body such as a skirt can extend between the dispenser arms 98. Further, the dispenser arms can comprise grooves or rails in some applications. Other forms of retaining the anchors can be utilized as desired. In some applications, other forms of actuation mechanisms and dispensing mechanisms can be utilized as desired. Other modifications can be provided as desired.

The anchor dispensing apparatus can be utilized solely according to applications. As such, the anchors 10 disclosed and other components of the anchoring system can be utilized separately from the anchor dispensing apparatus. The dispensing head of the anchor dispensing apparatus can further be utilized separate from the shafts and other components of the anchor dispensing apparatus.

FIGS. 21-25 illustrate a method that can utilize the anchor dispensing apparatus 88 and the anchors 10. The method is exemplary, and features can be added to, subtracted, or substituted across implementations as desired. The method is shown and discussed in regard to accessing and treating a mitral heart valve, however, the systems, apparatuses, and methods disclosed herein may be utilized to treat other native heart valves, such as tricuspid heart valves, or other features of a patient's anatomy mutatis mutandis. The method can comprise beating heart repair of a valve, including a mitral or tricuspid valve, among other valves. The method as disclosed can be transcatheter and percutaneous, however, more invasive methods such as a sternotomy or other methods of accessing the treatment site may be utilized in some applications as desired. The method can comprise an annular reduction method for regurgitation treatment according to applications herein. The shape of the annulus can be reshaped or reconstructed according to applications herein. The method(s) and steps herein can be performed on a living animal or on a simulation, such as on a cadaver, cadaver heart, simulator (e.g., with the body parts, heart, tissue, etc. being simulated), etc.

Referring to FIG. 21, the anchor dispensing apparatus 88 can be passed into the patient's body 126 via a percutaneous entry. The elongate shaft 90 of the apparatus 88, for example, can be passed through the vasculature of the body, such as a femoral artery 128. The dispensing head 96 can be covered with a sheath during initial entry into the patient's body or can be exposed during entry. A user can operate a housing, such as a handle 130 at the proximal end of the elongate shaft 90, to navigate the elongate shaft 90 to the desired implantation site.

The path of the elongate shaft 90 to the patient's heart, or other implantation site, can be viewed with medical imaging devices, including fluoroscopy and/or echocardiography as desired.

The elongate shaft 90 can pass transvascularly and transcatheterly into the patient's heart. In some applications, the elongate shaft 90 is navigated transfemorally and through the inferior vena cava into the right atrium 132 of the patient's heart. Other approaches are also possible, e.g., via the superior vena cava, etc. In some applications in which the tricuspid valve is treated, the elongate shaft 90 can deflect in order to position the dispensing head 96 proximate the tricuspid valve and within the right atrium 132. In some applications in which the mitral valve is to be treated, a transseptal puncture can be made between the right atrium 132 and the left atrium 134 to position the dispensing head 96 proximate the mitral valve and within the left atrium 134. Other access methods can be utilized, including transjugular access or transapical access, among others.

The anchor dispensing apparatus 88 can be positioned proximate the patient's native heart valve. The steerable shaft 112 can be operated to deflect the dispensing head 96 into a desired orientation relative to the mitral valve.

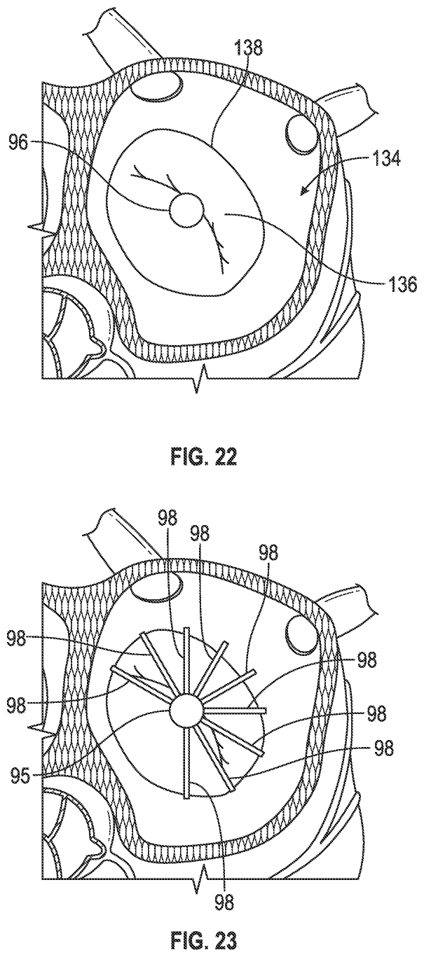

FIG. 22, for example, illustrates a top schematic view of the dispensing head 96 positioned relative to the mitral valve 136. The dispensing head 96 can be positioned to extend axially with the mitral valve 136 and can be centered over the mitral valve 136 or at another desired location. Similarly, the dispensing head 96 can be positioned over the tricuspid valve, or another valve to be treated as desired. The dispensing head 96 can be positioned on the atrial side of the valve leaflets or can be advanced distally to be positioned within the annulus as desired.

A native valve may suffer from a variety of maladies that may make it desirable to repair the native valve. For example, the annulus of the native valve may have dilated, causing valve regurgitation or other maladies. The systems, methods, and apparatuses disclosed herein can be utilized to repair the native valve and can be utilized to reshape or contract the annulus to a desired amount. Treatment of the mitral valve can be similar to that described and shown herein. In an application in which the tricuspid valve is treated, a similar operation or method to that treating the mitral valve can be used to treat maladies of the tricuspid valve, including reshaping or contracting the annulus of the tricuspid valve.

The dispensing head 96 can be navigated into position proximate the native valve with the dispenser arms 98 in the undeployed configuration and extending axially relative to each other, for example as shown in FIG. 10.

With the dispensing head 96 in position relative to the native valve, the actuation mechanism can be operated to expand the dispenser arms 98 radially outward. The dispenser arms 98 can be pivoted from the undeployed configuration (for example, as shown in FIG. 10) radially outward to the deployed configuration (for example as shown in FIG. 18). FIG. 23, for example, illustrates a top schematic view of the dispenser arms 98 expanded radially outward. The distal ends of the dispenser arms 98 can be circumferentially spaced from each other at a desired amount, for example, an equal amount or another amount as desired. The distal ends may be circumferentially spaced equally from each other when the dispenser arms extend radially outward from the central portion 95. An angle between the distal ends of the dispenser arms 98 can be equal or can be at a different amount. Further the radial distance of the distal ends of the dispenser arms 98 from the central portion 95 of the anchor dispensing apparatus 88 can be the same or can vary. For example, as represented in FIG. 20, if one of the dispenser arms 98 contacts a wall of the atrium, the radial expansion of that arm 98 can cease while the expansion of other arms can continue.

Notably, the dispenser arms 98 can be positioned circumferentially about only a portion of the central portion 95 of the anchor dispensing apparatus 88, in a desired position for dispensing the anchors 10. In some applications however, the dispenser arms 98 can be positioned circumferentially about the entirety of the central portion 95 of the anchor dispensing apparatus 88 or can otherwise be positioned according to the desired placement of the anchors.

A user can view the position of the dispenser arms 98 using medical imaging, such as fluoroscopy and/or echocardiography, to confirm the position of the dispenser arms 98 including the distal ends of the dispenser arms 98.

With the dispenser arms 98 in the desired position, the anchors 10 can be dispensed from the dispenser arms 98 to the tissue surrounding the patient's native heart valve. The dispensing mechanism including one or more push bodies 110, for example, can be utilized to dispense the anchors 10 as shown in FIG. 17. The push bodies 110 can push the anchors 10 distally from the plurality of dispenser arms 98.

In some applications, the anchors 10 can be dispensed from all of the dispenser arms 98 simultaneously to the tissue surrounding the patient's native heart valve or, in some applications, can be dispensed sequentially. A combination of simultaneous and sequential dispensing can be utilized in some applications. A user can control movement of the push body 110 to either simultaneously or sequentially dispense the anchors 10 as desired. An application in which the anchors 10 are simultaneously dispensed can reduce the number of steps in the treatment procedure and can reduce variability associated with sequentially dispensing the anchors 10. A user can selectively control the push bodies by controlling the movement of the proximal portions of the push bodies outside the patient's body, or in another manner.

The anchors 10 can deploy within the patient's tissue to the deployed configuration shown in FIG. 1. The anchors 10 can self-expand to the deployed configuration shown in FIG. 1 upon being pushed distally from the plurality of dispenser arms 98.

The anchors can be spaced from each other about the periphery of the patient's native heart valve. The anchors can be positioned in a curve about the patient's native heart valve. The anchors can be positioned in a desired manner to reshape the annulus as desired.

With the anchors 10 dispensed and moved into an expanded state as shown in FIG. 5, the spacer bodies 34 can rotate to extend between adjacent anchors 10. FIG. 24, for example, illustrates the anchors 10 implanted about the periphery of the mitral valve annulus with the spacer bodies 34 extending between the anchors 10. The contraction member 46 can extend through the spacer bodies 34, and the proximal portion 58 of the contraction member 46 can be accessible to tension and/or contract the implant.

The one or more contraction members 46 coupled to the plurality of anchors 10 can be tensioned to cinch the plurality of anchors 10. The contraction member 46 can extend from the distal anchor to one or more intermediate anchors to the proximal anchor 60. The contraction member 46 can be tensioned at the proximal anchor 60.

In some applications, a lock 64 can be advanced distally along the contraction member 46 and can be advanced via use of a tensioning mechanism as discussed in regard to FIGS. 7-9. The tensioning mechanism can advance the lock 64 distally to the proximal anchor 60 and can cinch, tension, and/or contract the contraction member 46 in a manner discussed in regard to FIGS. 7-9. In some applications, other forms of tensioning can be utilized, e.g., a spool can be included with the implant that can be rotated in a first direction to take up the contraction member and in a second direction to release the contraction member.

In some applications, the anchors 10 can be cinched/tensioned/contracted with the user monitoring blood flow and/or pressure through the native valve and monitoring the tension in the contraction member 46.

The distance between the anchors 10 can be reduced and the native valve annulus accordingly can be contracted by the movement of the anchors 10 or pulling them closer together. FIG. 25, for example, illustrates the movement of the anchors 10 radially inward towards the mitral valve 136 thus reducing the diameter of the mitral valve annulus 138.

A lock 64 can be utilized to lock the one or more contraction members 46 in a tensioned configuration. The lock 64 can maintain the contraction member 46 in a tensioned state and a cutting mechanism can cut the proximal portion of the contraction member 46 as discussed in regard to FIG. 9. The anchors 10 can remain in place surrounding the native valve. The anchor dispensing apparatus 88 and tensioning and cutting mechanisms can be removed from the patient's body.

The one or more contraction members 46 can be tensioned to cinch the anchors 10 to reduce a size of the annulus of the native heart valve. Such tensioning can be utilized to treat maladies of the heart valve including regurgitation of the heart valve, among other maladies.

The method, systems, and apparatuses disclosed herein can be modified as desired. For example, the position of the anchors 10 can be varied as desired. One or more anchors 10, for example, can be positioned on a leaflet of the mitral valve or another position within the atrium or patient's heart or body as desired. In an application in which tricuspid repair is provided, the anchors 10 can surround the tricuspid valve. Other locations of implantation can be utilized as desired. PCT Patent Application No. PCT/IB2020/060044, which is incorporated by reference herein in its entirety for all purposes, describes additional systems, apparatuses, methods, steps, features, components, designs, etc. that can be used and/or substituted in this application, e.g., anchors, contraction members, spacers, contraction mechanisms, locks, cutting mechanisms, tools, and/or other features that can be used.

The use of the anchor dispensing apparatus 88 can provide a variety of benefits, including simultaneous dispensing of a plurality of anchors circumferentially spaced from each other, and reducing the number of steps associated with dispensing a plurality of anchors. Further, the anchor dispensing apparatus 88 may not need to be navigated separately to each dispensing point of the anchors 10, thus reducing the amount of navigation required of the dispensing head and reducing the complexity of the procedure.

In addition, the number of anchors 10 utilized for the procedure can be reduced from other forms of valve repair, as the double curved hook shape of the anchor arms can enhance the anchoring of the anchors 10 to the patient's tissue.

In some applications, the configuration of the components of the systems can be varied as desired. For example, the form of the anchors 10 can be varied to comprise single shaft barbs, or helixes or other forms. Further, the configuration of the spacers can be varied as desired. For example, the spacers may not be connected to the anchors 10 in some applications and may be able to freely slide upon the contraction member between the anchors 10. Further, the number of anchors 10 and dispenser arms can be varied in some applications as desired.

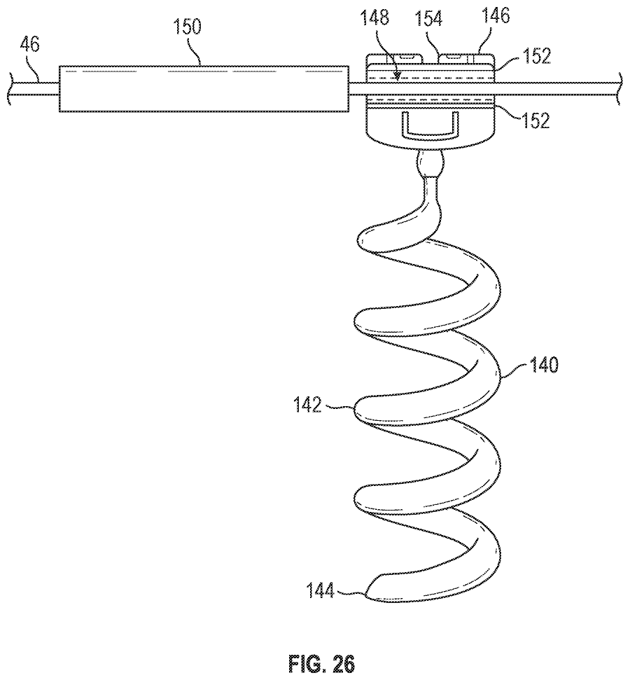

FIG. 26, for example, illustrates an application of an anchor 140 that can be utilized according to applications herein. The anchor 140 can include a distal portion 142 including a helix shape with a puncturing tip 144 at the distal end of the anchor 140. The anchor 140 can include a rotatable body 146 at a proximal portion of the anchor 140. The anchor 140 can further include a channel 148 configured to receive a contraction member 46 in a similar manner as the channel 44 shown in FIG. 4. The length of the distal portion can be between 8 millimeters and 12 millimeters and can be about 9 millimeters in some applications, or a greater or lesser amount. The diameter of the distal portion can be between 2 millimeters and 3 millimeters and can be about 2.5 millimeters in some applications, or a greater or lesser amount. Other dimensions can be utilized as desired.

The spacer body 150 between adjacent anchors 140 can be able to freely slide upon the contraction member 46 between adjacent anchors 140 and can contact the proximal portion of the anchor 140 or an adjacent spacer body 150 to space the adjacent anchors 140.

The rotatable body 146 can be configured to rotate free of the rotation of the channel 148, and thus can couple to the channel 148 at a pivot 152 or the like. As such, the rotatable body 146 can rotate in order to rotate the distal portion 142, without rotation of the channel 148. A portion of a dispensing mechanism can engage a coupling portion 154 of the rotatable body 146 to cause the distal portion 142 to rotate without rotating the channel 148. For example, a dispensing mechanism including an anchor driver can engage the coupling portion 154 to rotate the helix shaped distal portion 142 without rotating the channel 148.

The anchor 140 can be deployed by the dispenser arms 98 disclosed herein and can slide along the channels of the dispenser arms 98. The dispensing mechanism can push and engage the coupling portion 154 to rotate the rotatable body 146 to anchor the distal portion 142 into engagement with the tissue. The anchor 140 can be spaced from adjacent anchors via the spacer body 150 and can operate in a similar manner as the other anchors disclosed herein. Other variations of anchors can be utilized according to applications herein.

The “user” as discussed herein can comprise a user of the systems and apparatuses disclosed herein, which can include a surgeon, or another individual such as a medical professional who may operate the systems and apparatuses disclosed herein, without limitation.

The apparatuses and other devices disclosed herein can be practiced separately as desired. In addition, the methods herein are not limited to the methods specifically described, and can include methods of utilizing the systems, apparatuses, and devices disclosed herein.

In closing, it is to be understood that although aspects of the present specification are highlighted by referring to specific implementations, one skilled in the art will readily appreciate that these disclosed implementations are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular implementations only and is not intended to limit the scope of systems, apparatuses, and methods as disclosed herein, which is defined solely by the claims. Accordingly, the systems, apparatuses, and methods are not limited to that precisely as shown and described.

Certain implementations of systems, apparatuses, and methods are described herein, including the best mode known to the inventors for carrying out the same. Of course, variations on these described implementations will become apparent to those of ordinary skill in the art upon reading the foregoing description. The inventor expects skilled artisans to employ such variations as appropriate, and the inventors intend for the systems, apparatuses, and methods to be practiced otherwise than specifically described herein. Accordingly, the systems, apparatuses, and methods include all modifications and equivalents of the subject matter recited in the claims appended hereto as permitted by applicable law. Moreover, any combination of the above-described implementations in all possible variations thereof is encompassed by the systems, apparatuses, and methods unless otherwise indicated herein or otherwise clearly contradicted by context.

Groupings of alternative implementations, elements, or steps of the systems, apparatuses, and methods are not to be construed as limitations. Each group member may be referred to and claimed individually or in any combination with other group members disclosed herein. It is anticipated that one or more members of a group can be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses an approximation that can vary, yet is capable of performing the desired operation or process discussed herein.

The terms “a,” “an,” “the” and similar referents used in the context of describing the systems, apparatuses, and methods (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. Further, the techniques, methods, steps, etc. described or suggested herein or in these incorporated references can be performed on a living animal or on a non-living simulation, such as on a cadaver, cadaver heart, simulator (e.g., with the body parts, tissue, etc. being simulated), etc. The use of any and all examples, or exemplary language (e.g., “such as”) provided herein is intended merely to better illuminate the systems, apparatuses, and methods and does not pose a limitation on the scope of the systems, apparatuses, and methods otherwise claimed. No language in the present specification should be construed as indicating any non-claimed element essential to the practice of the systems, apparatuses, and methods.

All patents, patent publications, and other publications referenced and identified in the present specification are individually and expressly incorporated herein by reference in their entirety for the purpose of describing and disclosing, for example, the compositions and methodologies described in such publications that might be used in connection with the systems, apparatuses, and methods. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

What is claimed is:

1. An anchoring system comprising:
a plurality of anchors each configured to anchor to tissue of a patient's body;
one or more contraction members configured to couple the plurality of anchors to each other and to cinch the plurality of anchors;
a plurality of spacer bodies engaging the one or more contraction members and configured to space the plurality of anchors from each other when the plurality of anchors are anchored to the tissue; and
an anchor dispensing apparatus having a central portion and including a plurality of dispenser arms circumferentially spaced from each other and each configured to extend radially outward from the central portion to a distal end of the respective dispenser arm, each dispenser arm configured to dispense at least one of the plurality of anchors to the tissue of the patient's body.

2. The anchoring system of claim 1, wherein the central portion includes a central support, and each of the plurality of dispenser arms includes a proximal portion coupled to the central support.

3. The anchoring system of claim 1, wherein each of the plurality of dispenser arms is configured to pivot from an undeployed configuration radially outward to a deployed configuration.

4. The anchoring system of claim 3, further comprising an actuation mechanism configured to pivot each of the plurality of dispenser arms from the undeployed configuration radially outward to the deployed configuration.

5. The anchoring system of claim 4, wherein the actuation mechanism includes:
a plurality of support arms each coupled to one of the plurality of dispenser arms;
a first shaft coupled to proximal portions of the plurality of dispenser arms; and
a second shaft coupled to proximal portions of the plurality of support arms and configured to move relative to the first shaft to pivot each of the plurality of support arms radially outward.

6. The anchoring system of claim 1, wherein the distal end of each of the dispenser arms is configured for the at least one of the plurality of anchors to be dispensed from.

7. The anchoring system of claim 1, wherein the distal end of each of the dispenser arms includes an opening for the at least one of the plurality of anchors to be dispensed from.

8. The anchoring system of claim 1, wherein each of the plurality of anchors is configured to move from an undeployed configuration to a deployed configuration and has a linearized shape in the undeployed configuration and an expanded shape in the deployed configuration.

9. The anchoring system of claim 8, wherein each of the plurality of anchors is configured to self-expand from the undeployed configuration to the deployed configuration.

10. The anchoring system of claim 1, wherein each of the plurality of anchors has at least two anchor arms coupled together at a central portion of the anchor and each extending to a tip of the respective anchor arm.

11. The anchoring system of claim 10, wherein the at least two anchor arms are configured to form curved hooks extending outward from each other.

12. The anchoring system of claim 1, wherein the plurality of spacer bodies are configured to extend over the one or more contraction members.

13. The anchoring system of claim 1, wherein each of the plurality of spacer bodies are configured to pivotally couple to one of the plurality of anchors.

14. The anchoring system of claim 1, wherein the plurality of spacer bodies are each configured to extend axially relative to one of the plurality of anchors when the respective one of the plurality of anchors is in an undeployed configuration.

15. The anchoring system of claim 1, wherein each of the plurality of dispenser arms includes a channel configured to retain the at least one of the plurality of anchors and at least one of the plurality of spacer bodies.

16. The anchoring system of claim 1, further comprising a tensioning mechanism for tensioning the one or more contraction members.

17. The anchoring system of claim 1, further comprising a lock for locking the one or more contraction members in a tensioned configuration.

18. The anchoring system of claim 17, further comprising a cutting mechanism for cutting the one or more contraction members in the tensioned configuration.

19. The anchoring system of claim 1, further comprising a dispensing mechanism for dispensing each of the plurality of anchors from the plurality of dispenser arms, the dispensing mechanism including one or more push bodies configured to push the plurality of anchors distally from the plurality of dispenser arms.

20. The anchoring system of claim 1, wherein the anchor dispensing apparatus is configured to dispense the plurality of anchors to tissue that surrounds a mitral heart valve or a tricuspid heart valve.
